(19) United States
(12) Patent Application Publication
(10) Pub. No.: US 2022/0031481 A1
(43) Pub. Date: Feb. 3, 2022

(54) Intraluminal Stent and Preparation Method Therefor

(71) Applicant: Micro-Tech (Nanjing) Co., Ltd., Nanjing (CN)

(72) Inventors: Derong Leng, Nanjing (CN); Yuqian Li, Nanjing (CN); Nianci Xu, Nanjing (CN); Yongxue Zhao, Nanjing (CN); Changwang Pan, Nanjing (CN); Ning Li, Nanjing (CN); Chunjun Liu, Nanjing (CN); Zhi Tang, Nanjing (CN); Hongyan Jin, Nanjing (CN); Chen Zhang, Nanjing (CN)

(21) Appl. No.: 17/503,700

(22) Filed: Oct. 18, 2021

Related U.S. Application Data
(63) Continuation-in-part of application No. PCT/CN2019/126080, filed on Dec. 17, 2019.

(30) Foreign Application Priority Data
May 28, 2019 (CN) 201910453118.2

(51) Int. Cl. A61F 2/852; A61F 2/88

(52) U.S. Cl. CPC A61F 2/852; A61F 2/885; A61F 2210/0066; A61F 2230/0091; A61F 2230/0034; A61F 2230/0043; A61F 2230/006; A61F 2210/0076

(57) ABSTRACT

An intraluminal stent (1) and a preparation method therefor are provided. The intraluminal stent (1) includes at least one sub-stent. The sub-stent includes at least a first wire (4) extending along a first spiral direction and at least a second wire (5) extending along a second spiral direction, and the first wire (4) and the second wire (5) extend in different directions to form several wire intersection points. The intraluminal stent (1) has several wrapped portions (2) to wrap two corresponding wire tail ends. A wrapping connector (3) is disposed at a periphery of the wrapped portion (2). Two end portions of the wrapping connector (3) are firmly connected to regions corresponding to two ends of the wrapped portion (2). The two wire tail ends of the wrapped portions (2) are wrapped by using the wrapping connector (3). The stent (1) is less harmful to intraluminal tissues and a relatively strong tensile resistance.

This application claims the priority to the Chinese Application No. 201910453118.2, filed with the Chinese Patent Office on May 28, 2019 and entitled “INTRALUMINAL STENT AND PREPARATION METHOD THEREFOR”, which is incorporated herein by references in its entirety.

### FIELD OF THE INVENTION

This application relates to an intraluminal stent used for human body lumen, which belongs to a medical device used in human body.

### BACKGROUND OF THE INVENTION

Existing stent braiding methods are mainly classified into manual braiding and machine braiding. The manual braiding is difficult in braiding and has a limited production capacity. For the machine braiding, it is difficult to process a stent head, and usually an end of a fine wire and a stent body cannot be well fixed, and as a result, a connection portion of the stent is easy to be broken. For example, in the prior art CN105873547A, a stent is prepared by means of machine braiding. In one aspect, an end connection portion formed in an end portion re-braiding region of a wire is close to edges at both ends of the stent. During a tensile process, the connection portion is stressed to yield, having relatively worse deformability and tensile resistance performance. As a result, it is easy for the stent to damage tissue in the lumen, and the stent is very easy to be broken, making it difficult to take out the stent. In another aspect, a welding process is used for the connection portion, making a surface of a connection point rough, thus easily damaging lumen tissue. Moreover, anti-tensional strength is poor if an area of a welding point is small; and rigidity of a connection portion is excessively great if an area of a welding point is excessively large. In this case, environmental adaptability in the lumen and deformability are reduced. In addition, it may be learned from patents US20050256563A1 and US20170231746A1 that during a stent braiding process, in a re-braiding manner generally used in the prior art, processing methods for a connection portion are similar to that in CN105873547A. Therefore, the above-mentioned technical problems that are difficult to be overcome also exist, for example, an intraluminal tissue is seriously damaged, and the connection portion has poor tensile resistance performance.

In addition, in the prior art CN107595448A, although an outer portion of a wire end uses a tube structure, a tail end of the wire end is still fixedly connected. In other words, two tail ends of the wire end are fixedly connected and then an outer tube structure is sleeved thereto. A biggest defect of such wire end connection manner, found by researchers, is that during a tensile process, when a radial or axial extraction force that exceeds tolerance limit of a wire-end connection portion is applied, the abrupt deformation of the stent at the moment when the wire end fractures has relatively great impact on the tissue and bring in great damages to the tissue. In addition, a wire-end connection section in the prior art is also disposed at an edge portion of the stent, and a sharp corner may be formed during a process in which the wire end fractures, thus increasing a probability of damaging human tissue.

In view of the above, this application is hereby proposed.

### SUMMARY OF THE INVENTION

On the basis of the technical problem existing in the prior art, an intraluminal stent that is less harmful to intraluminal tissues and has an outstanding tensile resistance is provided, including an intraluminal stent, wherein the intraluminal stent includes at least one sub-stent; the sub-stent has a radial compression form and a radial expansion form; the sub-stent has, in an axial direction, end portion regions at two ends and an intermediate region extending between the end portion regions; the sub-stent includes at least a first wire extending along a first spiral direction and at least a second wire extending along a second spiral direction; the first wire and the second wire extend in different directions to form several wire intersection points; the intraluminal stent has several wrapped portions for wrapping two corresponding wire tail ends; a wrapping connector is disposed at a periphery of the wrapped portion; two end portions of the wrapping connector are firmly connected to regions corresponding to two ends of the wrapped portion; and the wrapped portion is disposed at a distance from an edge of at least one end portion of the sub-stent. By disposing the wrapped portion to be away from the edge of the end portion of the intraluminal stent (the sub-stent) for a distance, compared with an existing manner of directly disposing a wire-end connection portion at an edge or at a position adjacent to the edge, the disposing manner in this application makes freedom of the wire-end connection portion be limited by a braided mesh of the stent. In this way, the stent is not easy to be deformed and fail, and radial compression resistance force and extraction force thereof are improved, so that mechanical properties of the stent can be significantly improved.

Further, a separation distance between the wrapped portion and the edge of at least one end portion of the stent is a, and a length of the sub-stent along the axial direction is L, where a/L is greater than 1/20.

Further, the two wire tail ends of the wrapped portions are wrapped by using the wrapping connector in a manner of being close to or in contact with each other.

Further, the two wire tail ends of the wrapped portions are wrapped by using the wrapping connector in a manner that the two tail ends can move relative to each other.

Further, radial compression resistance force of a corresponding portion of the wrapping connector is not smaller than 10 N.

Further, the wrapping connector is an elastic coating layer and/or a heat-shrinkable tube.

Further, a length of the wrapping connector at least completely covers the wrapped portion, and the two corresponding wire tail ends in the wrapped portion are in contact with each other in an abutting manner or have an overlapping region.

Further, at least one end portion region of the stent contracts radially when closing to the intermediate region, the wrapped portion is located at a radial contraction portion, and the wrapping connector is located at a radial contraction region.

Further, a cross section of the sub-stent is circular, D-shaped, or elliptical.

Further, the intraluminal stent is an L-shaped or a Y-shaped stent.

Further, the tail end of the first wire extending along the first spiral direction is bent towards the intermediate region at the end portion region on at least one end of the sub-stent to form a bent portion, and is close to the corresponding tail end of the second wire extending along the second spiral direction to form a wrapped portion.

Further, between two sub-stents, a wire tail end of one sub-stent extends towards a corresponding wire tail end of another sub-stent to form a wrapped portion.

This application further provides a method for preparing an intraluminal stent, including the following steps:

(1) providing an intraluminal stent that includes at least one sub-stent;

(2) making two corresponding wire tail ends in the sub-stent close to each other to form a wrapped portion, wherein several wrapped portions are formed on the sub-stent; and

(3) disposing a wrapping connector at a periphery of the wrapped portion, enabling two end portions of the wrapping connector to be firmly connected to regions corresponding to two ends of the wrapped portion, wrapping two wire tail ends of the wrapped portions by using the wrapping connector, and enabling the wrapped portion to be disposed at a distance from an edge of at least one end portion of the sub-stent.

Further, in step (3), the two wire tail ends penetrate into a wire mesh formed by a first wire extending along a first spiral direction and at least a second wire extending along a second spiral direction to form the wrapped portion.

Further, in step (2), the two end portions of the wrapping connector are fixed at the regions corresponding to the two ends of the wrapped portion through a coating process or a heat shrinking process to form firm connections.

On the basis of the foregoing technical solutions, this application has the following advantages.

The stent in this application uses a multi-wire stent braided by a machine with higher productivity as a body, and uses a connection manner of a non-welded wire end portion (a wire end), so that the end portions of both ends of the stent are as smooth as that of a conventional one-wire stent that is manually braided. Thus, the stent in this application has little irritation to a tube wall, reduces risks of hyperplasia, perforation, and displacement, and also reduces a risk that the connector drops during a position adjustment and recovery of the stent.

In addition, by optimizing the position of the wrapped portion on the stent, under a premise of ensuring tensile performance of the stent, tensile capability at a connection position of the wire end is improved, thereby ensuring that the stent is not easy to be broken during use, and improving safety performance during use of the stent.

### BRIEF DESCRIPTION OF THE DRAWINGS

FIG. 1a and FIG. 1b are side views when wrapping connectors in an intraluminal stent extend along different spiral directions;

FIG. 2 is a schematic diagram of an end of an intraluminal stent that is applied with axial force;

FIG. 3 is a schematic diagram of the intraluminal stent shown in FIG. 2 that is applied with radial force;

FIG. 4a to FIG. 4c are enlarged schematic diagrams of different manners in which corresponding regions of a wrapping connector of the intraluminal stent shown in FIG. 3 are close to each other;

FIG. 5 is a schematic diagram of a radial cross section of an intraluminal stent;

FIG. 6 is a schematic diagram of a radial cross section of an intraluminal stent;

FIG. 7 is a schematic diagram of a radial cross section of an intraluminal stent;

FIG. 8a to FIG. 8f are schematic diagrams of an L-shaped stent composed of two sub-stents, wherein FIG. 8a to FIG. 8f show different arrangement manners of a wrapping connector in an intraluminal stent on different sub-stents; and

FIG. 9a to FIG. 9l are schematic diagrams of a Y-shaped stent composed of three sub-stents, wherein FIG. 9a to FIG. 9l show different arrangement manners of a wrapping connector in an intraluminal stent on different sub-stents.

### REFERENCE NUMERALS IN THE DRAWINGS

1 Stent
11 End Portion Region
12 Intermediate Region
2 Wrapped Portion of the Stent
3 Wrapping Connector
4 First Wire
41 First Binding Point
5 Second Wire
51 Second Binding Point

### DETAILED DESCRIPTION OF THE EMBODIMENTS

Technical content of this application is described in detail with reference to the accompanying drawings and specific embodiments.

Implementations of this application are described in detail below with reference to embodiments, but a person skilled in the art may understand that the following embodiments are merely for describing this application, and should not be regarded as limitation on the scope of this application.

Some terms involved in this application are explained hereinafter. A compressed form refers to a state when a stent is compressed radially during a delivery process of the stent. An expansion form refers to a form after the stent is released and is expanded due to own radial tension. An axial direction refers to an extension direction of central axis of various sub-stents in an intraluminal stent. A radial direction refers to a radial extension direction perpendicular to the axial direction. In addition, a first wire and a second wire are further explained. A first wire extending along a first spiral direction refers to all wires, in various sub-stents in an intraluminal stent, that extend along the first spiral direction, and is not limited to a certain wire. Similarly, a second wire extending along a second spiral direction refers to all wires, in various sub-stents in the intraluminal stent, that extend along the second spiral direction. According to specific forms of sub-stents in the intraluminal stent formed by means of machine braiding, the first wire and the second wire that extend in different spiral directions may be wire-braided objects that are relatively independent and separated from each other. In other words, both ends of the sub-stent in the intraluminal stent formed by means of machine braiding have wire end portions (wire ends) of a first wire extending along the first spiral direction and a second wire extending along the second spiral direction. The first wire and the second wire that extend along different spiral directions may be wire-braided objects formed after a same wire is entirely bent. In other words, only one end of the sub-stent in the intraluminal stent formed by means of machine braiding has end portions (wire ends) of a first wire extending along the first spiral direction and a second wire extending along the second spiral direction, while the other end does not have a wire end.

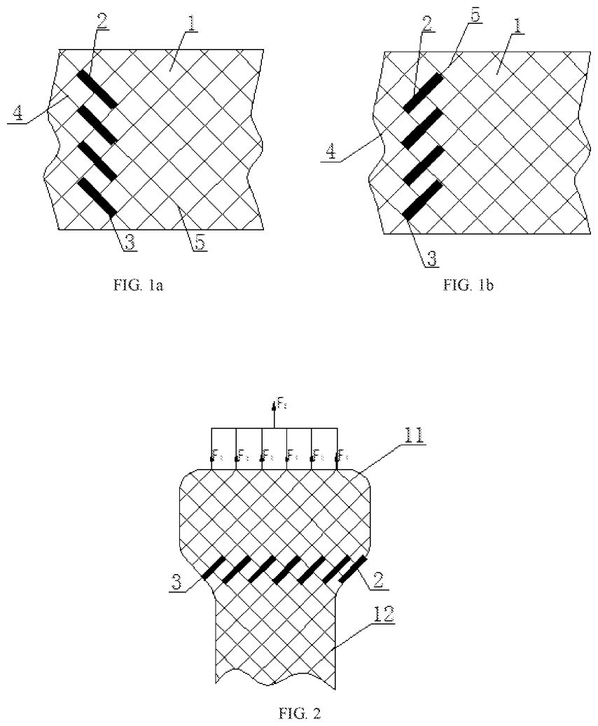

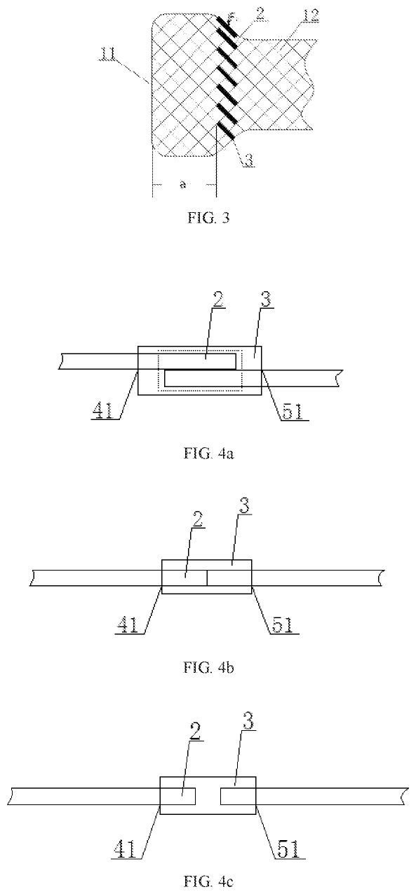

FIG. 1a, FIG. 2, and FIG. 3 show a corresponding region in a wrapping connector in at least one sub-stent in an intraluminal stent according to an embodiment of this application. The sub-stent is used in body lumen, and the body lumen may be pancreatic duct, bile duct, intestinal tract, esophagus, or trachea. The sub-stent has a radial compression form and a radial expansion form. The sub-stent has, in an axial direction, end portion regions 11 at two ends and an intermediate region 12 extending between the end portion regions 11. The sub-stent includes a plurality of first wires 4 extending along a first spiral direction and second wires 5 extending along a second spiral direction, wherein the quantity of the second wires 5 is corresponding to that of the first wires 4. The first spiral direction intersects with the second spiral direction, and the two are symmetrically disposed with respect to a central shaft of the stent. The first wire 4 and the second wire 5 extend to intersect with each other to be in a form of several meshes having several wire intersection points. FIG. 1b shows another implementation. The wrapping connector may extend along the first spiral direction. To be specific, the wrapping connector may extend along different spiral directions according to requirements of an actual braiding manner.

As shown in FIG. 2 and FIG. 3, a tail end of the first wire 4 extending along the first spiral direction is bent towards the intermediate region 12 at the end portion region 11 to form a bent portion, and overlaps and intersects with a tail end of the second wire 5 extending along the second spiral direction to form a wrapped portion 2. A tube-shaped wrapping connector 3 is disposed at a periphery of the wrapped portion. Two end portions of the wrapping connector 3 are firmly connected to regions corresponding to two ends of the wrapped portion 2. The tail ends of the first wire 4 and the second wire 5 of the wrapped portion 2 are wrapped in the wrapping connector 3 by using the wrapping connector 3. In this embodiment, a heat-shrinkable tube is preferably used to wrap the wrapped portion 2 at the wire tail end. A length of the heat-shrinkable tube that serve as a wrapping connector at least covers the entire length of the wrapped portion. The length of the heat-shrinkable tube is preferably at least 1 mm longer than end portions at both ends of the wrapped portion 2, to ensure that the wire tail end would not be pulled out of the tube during a tensile process, thereby improving stability of the wrapping connector 3. Certainly, in addition to the heat-shrinkable tube, an elastic coating layer may be formed at an external portion of an overlapping portion to cover the tail end of the wire. It should be noted that when the wrapping connector 3 is a heat-shrinkable tube, a heat shrinkage ratio of the heat-shrinkable tube is greater than or equal to 30%, so as to ensure that after heat shrinkage, the wrapping connector 3 that is a heat-shrinkable tube has relatively strong wrapping force. It should be noted that in actual application, the heat shrinkage ratio of the heat-shrinkable tube may be set according to actual requirements. For example, to ensure that tail ends of two wires wrapped by the heat-shrinkable tube can move relative to each other, the heat shrinkage ratio of the heat-shrinkable tube is set to be smaller than or equal to 80%.

As shown in FIG. 1a and FIG. 1b, a length of the wrapping connector 3 is twice of a width of a braided mesh; in another spiral direction, there is a first wire 4 or a second wire 5 that crosses the wrapping connector 3; the braided mesh is a grid formed by means that the first wire 4 intersects with the second wire 5; and said another spiral direction is a first spiral direction or a second spiral direction that is not provided with a wrapping connector 3.

When the length of the wrapping connector 3 is twice of the width of the braided mesh, both ends of the wrapping connector 3 are located at the wire intersection points of the first wire 4 and the second wire 5, and are clamped with the first wire 4 or the second wire 5 that is in said another spiral direction and forms the wire intersection points.

It should be noted that if the length of the wrapping connector 3 is longer, an overlapping region of two wire tail ends in the wrapping connector 3 may be set to be longer, and thus the wrapping connector 3 has stronger wrapping force to the wrapped portion 2. However, if the length of the wrapping connector 3 is greater than twice of the width of the braided mesh, the wrapping connector 3 needs to cross more wire intersection points. In this case, surface flatness of the sub-stent is affected, and wire stability in said another spiral direction is affected. When the length of the wrapping connector 3 is twice of the width of the braided mesh, while relatively strong wrapping force to the wrapped portion 2 is ensured, both ends of the wrapping connector 3 may be clamped with the first wire 4 or the second wire 5 that is in said another spiral direction and forms the wire intersection points, thereby preventing the wrapping connector 3 from moving relative to the wrapped portion 2, and further ensuring stability of the wrapped portion 2.

It should be noted that when the length of the wrapping connector 3 is twice of the width of the braided mesh, a length for which the two wire tail ends overlap with each other may be adaptively set to be twice of the width of the braided mesh. During a tensile process, because both ends of the wrapping connector 3 are clamped with the first wire 4 or the second wire 5 in said another spiral direction, it is ensured that the wrapping connector 3 does not move in a wire extension direction. In this way, the wire tail end would not be pulled out from the tube.

As shown in FIG. 2 and FIG. 3, to prevent the wrapping connector 3 from affecting the surface flatness of the sub-stent, in this embodiment, the wrapping connector 3 is located in a transition region between the end portion region 11 and the intermediate region 12 of the sub-stent. Because a radial diameter of the end portion region 11 is greater than that of the intermediate region 12, the transition region has an oblique angle with respect to the end portion region and the intermediate region 12. Therefore, surface flatness of the end portion region 11 and the intermediate region 12 is not affected.

FIG. 4a is an enlarged schematic diagram of a corresponding region of the wrapping connector 3. In this embodiment, the wrapping connector 3 completely covers the wrapped portion 2, and a length of the wrapped portion in an extension direction of the wire is greater than 3 mm One end of the wrapping connector 3 is stably bonded to the first wire at a first binding point. The other end of the wrapping connector 3 is stably bonded to the second wire at a second binding point. The wrapping connector 3 is elastic. Therefore, when the stent is dragged along an axial direction and the wrapped portion 2 is applied with axial tensile force, the first wire and the second wire are away from each other along the second spiral direction. In other words, the tail ends of the first wire and the second wire may move relative to each other (the two wire ends are close to each other but are not fixedly connected). Researchers of this application innovatively try to change a conventional fixed connection manner between wire ends into a relatively movable manner. Compared with the fixed connection manner between wire ends in the prior art, the arrangement in this application that the tail ends of the two wire ends may move relative to each other can greatly increase tensile flexibility of a connection point of the wire, and improve overall deformation performance of the stent. In this way, during deformation process of the stent, such as expansion and stretching, tensile resistance performance of the stent is significantly improved. More notably, different from the fixed connection manners in the prior art such as welding, a relatively stable wrapping connector is formed between the tail ends of the two wires by using a heat-shrinkable tube or a coating. According to such manner, in one aspect, it is ensured that an outer surface of a connection point is smoother, thus having little irritation to a duct wall, and reducing risks of hyperplasia, perforation, and displacement; and in another aspect, a risk of damaging human tissue by an instantaneous sudden change caused by fracture of the connector during a position adjustment and recovery of the stent is reduced. Certainly, except the case shown in FIG. 4a in which the two wire tail ends (wire ends) in the wrapped portion 2 have an overlapping region, as shown in FIG. 4b and FIG. 4c, the two wire tail ends may also be wrapped in the wrapping connector 3 in any manner of approaching each other. For example, the two wire ends may not be overlapped, and only the wire ends are in contact (as shown in FIG. 4b) or the wire ends are not in contact (as shown in FIG. 4c), provided that it is ensured that wrapped portion 2 is firmly connected by the wrapping connector 3.

Further, as shown in FIG. 2 and FIG. 3, radial compression resistance force of a corresponding portion of the wrapping connector 3 in the intraluminal stent is not smaller than 10 N. The radial compression resistance force refers to compression force required to radially compress the corresponding portion of the wrapping connector to be in a limit state (the wrapping connector is detached from the wire tail end). An edge portion of an end portion region of each sub-stent in the intraluminal stent has circumferential bent portions that surround circumferentially. Axial extraction force at an inflection point of a single bent portion is not smaller than 30 N. The axial extraction force is defined as pulling force that detaches a connection component from the wire tail end when a stent body is applied with axial force. A sum of axial extraction force at inflection points of bent portions of the stent body is not smaller than 200 N. On the basis of the improved wire connection manner in this application, during use and removal of the intraluminal stent, the intraluminal stent can satisfy the above requirements on compression resistance and tensile performance. Moreover, support of the stent to the lumen wall can further ensure that the stent is taken out of the human body lumen without damaging the tissue.

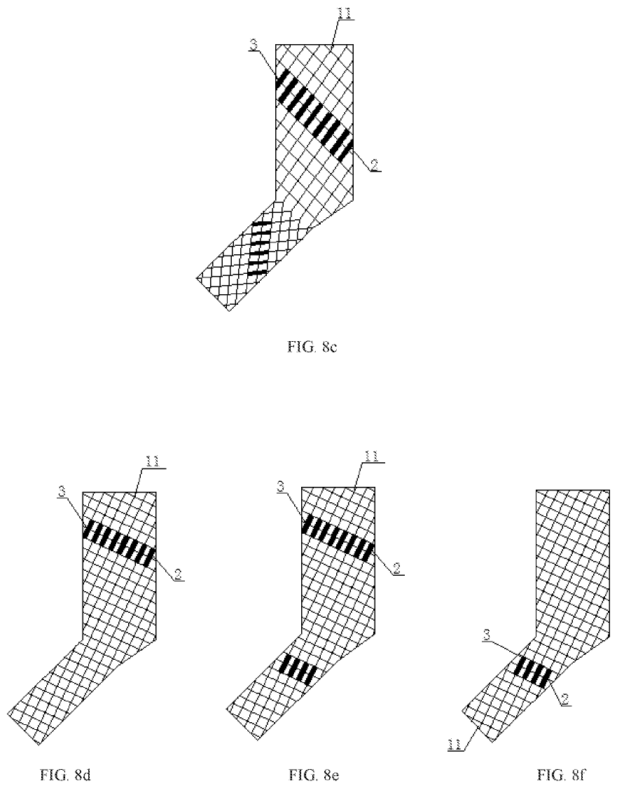

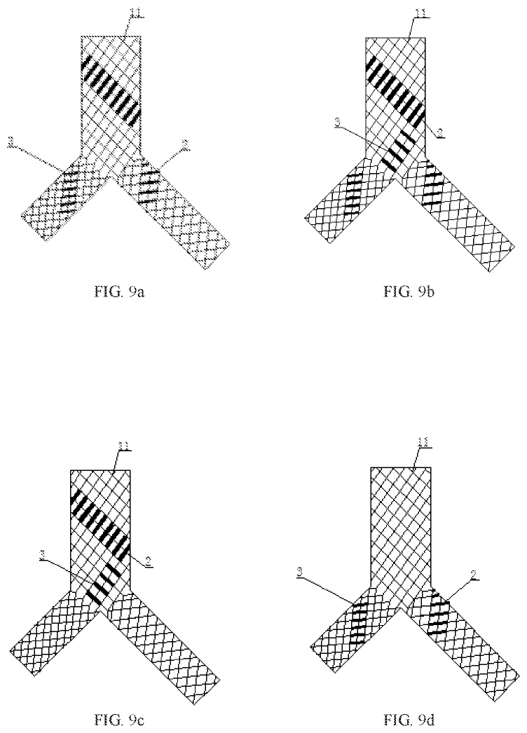

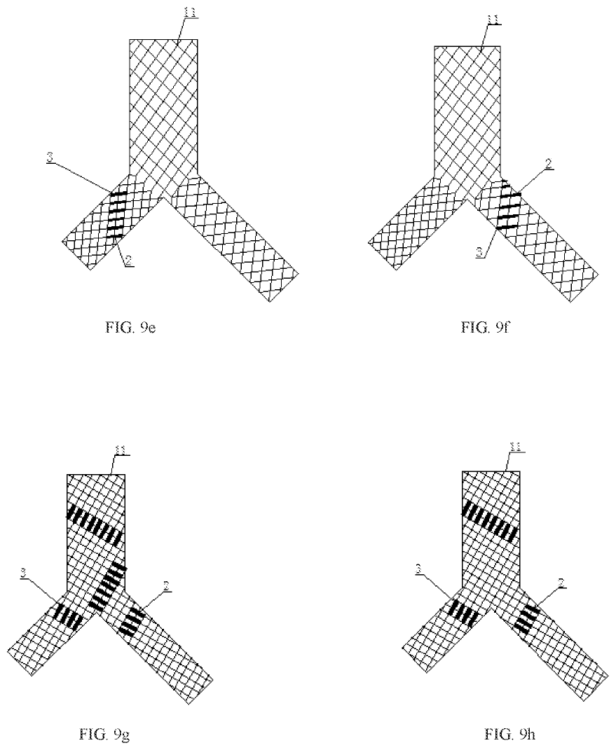

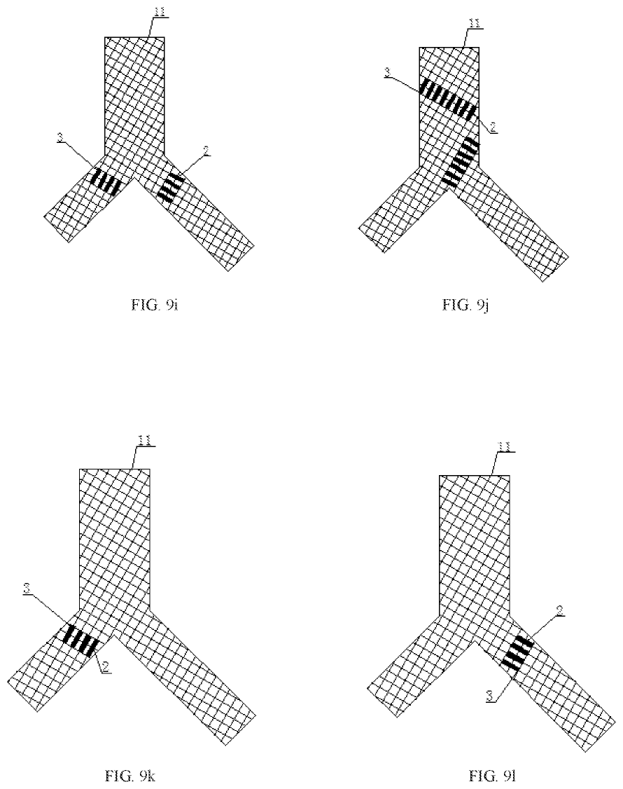

As shown in FIG. 2, FIG. 8a to FIG. 8f, and FIG. 9a to FIG. 9l, there is a distance from the wrapped portion 2 in the intraluminal stent to the edge of at least one end portion region of the sub-stent. Different from a conventional design in the prior art that a wire-end connection point is located at an edge of an end portion of the stent, in this application, it is preferable that the wrapped portion 2 corresponding to a wire-end connection point is away from the edge of the end portion of the stent. If it is set that a distance from a side, of the wire-end connection point (the wrapping connector), that faces the edge to the edge of the end portion of the stent is a, and an overall length of the sub-stent in the axial direction is L, it is preferable in this application that a/L is greater than 1/20. After repeated tests, when a/L is greater than 1/20, there is a sufficient distance between the wire-end connection portion and an edge region. In this case, a probability of the wrapped portion being pulled and broken during a tensile process is obviously reduced, and pull-off deformation performance is also improved. From a perspective of wire intersection point, it is preferable that there are at least three wire intersection points between the wrapped portion and the edge of the end portion of the sub-stent, or it is preferable that there are at least five wire intersection points between the wrapped portion and the edge of the end portion of the sub-stent. According to this special braiding manner of keeping the wrapping connector 3 away from the bent portion, yield and deformation performance of the stent is greatly improved. In one aspect, a defect that the connection point is easily fractured due to stress concentration of the connection point during the tensile process may be avoided. In another aspect, through repeated trials and feedback of clinical use, a braiding manner that the connection point is offset to the intermediate region of the stent can effectively reduce damages of the wire-end connection portion to lumen tissue during the tensile process.

The tensile performance of the stent in this application is verified according to the following test example.

Test example:

Test device: tensile testing machine, corresponding apparatuses are used for different tests.

Test conditions for axial extraction force: an auxiliary apparatus is fixed on a clamp of the tensile testing machine, an auxiliary wire at one end of the intraluminal stent is hung to the auxiliary apparatus, the other end of the intraluminal stent is fixed to another clamp of the tensile testing machine, and maximum tensile force F. (maximum tensile force that can be withstood when the wire-end connection portion is broken (the wrapping connector is detached from the wire tail end)) is recorded during the test. The test is carried out at a speed of 200 mm/min, and a gauge length is determined based on total lengths of different test samples in free states. For principle of selecting parameters, refer to Appendix B of Chinese standard GB/T15812.1-2005.

Test conditions for radial compression resistance force: a to-be-tested sample is fixed on an opening by using a cylindrical fixing tool at a room temperature, a probe with a rectangular probe is enabled to pass through the sample until a connector is separated from a connected element, and maximum load during the test is recorded. Operation steps: adjusting a fixed mold or probe, so that an annular opening and the probe are in concentric positions; placing a test portion of the sample at an opening of a cylindrical fixed mold; fixing the sample; slowly lowering the probe to be in contact with the sample; moving the probe at a speed of 50 mm/min, until the wire-end connection portion on the intraluminal stent is broken (the wrapping connector is detached from the wire tail end); and recording maximum load when the wire-end connection portion is broken.

Test results are shown in Table 1 and Table 2.

TABLE 1
Comparative test results when the wire-end
connection portion is at different position

| | a = 0 | a = | | | | |
|---|---|---|---|---|---|---|
| | (located | 1/20 | | | | |
| | at the | (spaced | | | | |
| Position | edge of | about | | | | |
| of the | the end | two or | | | | |
| wire-end | portion | three wire | | | | |
| connection | of | intersection | a = | a = | a = | a = |
| portion | the stent) | points) | 1/15 | 1/10 | ⅕ | ⅓ |
| Radial | 6.7N | 10.1N | 14.3N | 16.7N | 17.1N | 17.6N |
| com- | | | | | | |
| pression | | | | | | |
| resistance | | | | | | |
| force | | | | | | |
| Axial | 21.4N | 23.6N | 27.5N | 34.2N | 40.5N | 47.4N |
| extraction | | | | | | |
| force at an | | | | | | |
| inflection | | | | | | |
| point of a | | | | | | |
| single bent | | | | | | |
| portion | | | | | | |
| Sum of | 144.7N | 165.2N | 189.4N | 210.4N | 248.5N | 300.1N |
| axial | | | | | | |
| extraction | | | | | | |
| forces at | | | | | | |
| inflection | | | | | | |
| points | | | | | | |
| of bent | | | | | | |
| portions | | | | | | |

Note: in table 1, it is set that a distance from a side, of the wire-end connection point (the wrapping connector), that faces the edge to the edge of the end portion of the stent is a (as shown in FIG. 3), and an overall length of the sub-stent in the axial direction is L.

On the basis of the test results shown in Table 1, it may be learned that overall mechanical performance of the stent may be greatly improved after the wire-end connection portion is disposed at a distance from the edge of the stent. Compared with a disposing manner in the prior art that the wire-end connection portion is directly located at the edge of the stent or is adjacent to the edge of the stent, the radial compression resistance force in this application is greater than 10 N, thereby greatly improving safety performance of the stent during a process of pulling and taking out the stent. Through clinical trials, it is proved that compared with the prior art, the stent in this application is more excellent in safety.

TABLE 2
Comparative test results for different
connection manners of wire ends

| | Wire ends | Wire ends can move relative |
|---|---|---|
| Connection manner | are fixedly | to each other (a connection |
| of wire ends | connected | manner in this application) |
| Radial compression | 8.1 N | 10.2 N |
| resistance force | | |

Note: in table 2, it is set that a distance from a side, of the wire-end connection point (the wrapping connector), that faces the edge to the edge of the end portion of the stent is a (as shown in FIG. 3), and an overall length of the sub-stent in the axial direction is L, wherein a/L= 1/20.

On the basis of the test results shown in Table 2, it may be learned that by using the manner that the wire ends can move relative to each other, the radial compression resistance force of the stent is obviously improved. Moreover, compared with disposing the wire-end connection portion at the edge of the end portion of the stent, after the wire-end connection portion is disposed at a distance from the edge, both radial compression resistance performance and axial tensile performance are significantly improved.

In addition, as another preferable manner, as shown in FIG. 2, at least one end portion region of the intraluminal stent or any sub-stent contracts radially when closing to the intermediate region, and the wrapped portion 2 is located at a radial contraction portion; and correspondingly, the wrapping connector 3 is located at a radial contraction region. In one aspect, this design manner effectively improves radial expansion strength of the end portion of the stent. Meanwhile, when requirements on radial compressive strength are satisfied, a braiding position of the wrapping connector is close to the intermediate region having a relatively smaller radial width, thus effectively reducing a probability that the connection points are in contact with lumen tissue of human body, and reducing a probability of damaging the tissue.

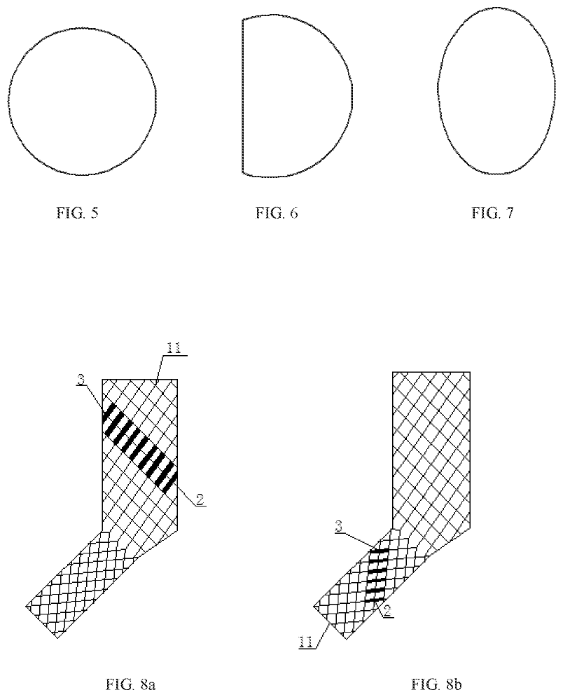

As shown in FIG. 5 to FIG. 7, a radial cross-section of the sub-stent body is preferably circular, D-shaped, or elliptical, to adapt to different tissue cavities. Different cross-sectional shapes meet requirements on strength in different directions. When the intraluminal stent includes a plurality of sub-stents, cross-sectional shapes of the sub-stents may be the same or different. Corresponding selections are made according to requirements.

In addition, as shown in FIG. 8a to FIG. 8f and FIG. 9a to FIG. 9l, when the intraluminal stent is composed of a plurality of sub-stents, an L-shaped stent composed of two sub-stents (as shown in FIG. 8a to FIG. 8f) may be formed, or a Y-shaped stent composed of three sub-stents (as shown in 9a to FIG. 9l) may be formed. A radial cross-section of each sub-stent may be circular, D-shaped, or elliptical. The wrapping connector 3 may be located on any one (as shown in FIG. 8a, FIG. 8b, and FIG. 8c) or more (as shown in FIG. 8c and FIG. 8e) of different sub-stents, and one or more wire-end connection regions (each wire-end connection region includes several wrapped portions 2 that are arranged in a same direction) may be formed on one sub-stent based on positions and a quantity of wire ends by using a corresponding braiding method. These wire-end connection regions may be located at any position on the axial direction of the intraluminal stent, and may extend along any spiral direction. As shown in FIG. 9a, FIG. 9d, FIG. 9e, FIG. 9f, FIG. 9h, FIG. 9i, FIG. 9k, and FIG. 9l, a sub-stent may be provided with only one wire-end connection region. In addition, as shown in FIG. 9b, FIG. 9c, FIG. 9g, and FIG. 9j, a sub-stent may be provided with a plurality of wire-end connection regions. In addition, in this application, it is preferable that the wrapped portion is disposed at a distance from the edge of the end portion of the stent, to ensure that an overall outside of the stent is smooth, and the tensile performance of the stent satisfies requirements for use in different cavities.

Specifically, on the basis of differences in the manner of machine braiding, a braiding manner of the wrapped portion 2 on the intraluminal stent may be correspondingly adjusted based on actual positions and quantity difference of wire ends. For example, when there is only one sub-stent in the intraluminal stent, the wire end of the first wire extending along the first spiral direction and the wire end of the second wire extending along the second spiral direction may be disposed at one end or both ends of the sub-stent. To form an enclosed end portion region of the stent, the tail end of the first wire (a wire end) extending along the first spiral direction may be bent towards the intermediate region at the foregoing end portion region of the sub-stent to form a bent portion, and is close to the corresponding tail end of the second wire extending along the second spiral direction to form a wrapped portion 2. Moreover, a corresponding wrapping connector 3 is disposed. In this way, an intraluminal stent of which only one end has wire-end connection regions (each wire-end connection region includes several wrapped portions 2 that are arranged in a same direction) is obtained, or an intraluminal stent of which both ends have wire-end connection regions (each wire-end connection region includes several wrapped portions 2 that are arranged in a same direction) is obtained. When the intraluminal stent is connected and braided by using a plurality of sub-stents, between two of the sub-stents, a wire tail end of one sub-stent extends towards a corresponding wire tail end of the other sub-stent to form a wrapped portion; and the two wire tail ends are firmly connected by using the wrapping connector 3. In the L-shaped intraluminal stent shown in FIG. 8a to FIG. 8f, according to distribution of wire ends after the machine braiding, there are one or two wire-end connection regions (each wire-end connection region includes several wrapped portions 2 that are arranged in a same direction) on the entire intraluminal stent. In the Y-shaped intraluminal stent shown in FIG. 9a to FIG. 9l, there are two to four wire-end connection regions (each wire-end connection region includes several wrapped portions 2 that are arranged in a same direction) on the entire intraluminal stent.

In addition, in this application, radiopaque marks, such as tantalum marks may be provided on the stent according to actual needs and specific use requirements to facilitate positioning during use. In addition, a take-up line may be provided at the end portion region of at least one end of the stent to help take back the stent. The take-up line may be in a separate structure or in a structure integrally braided with the stent. At least the intermediate region of the stent is covered with a stent film, and a material of the film is preferably a degradable material, to reduce irritation to the human body.

A method for preparing the foregoing intraluminal stent in this application includes the following steps:

(1) providing an intraluminal stent that includes at least one sub-stent;

(2) making two corresponding wire tail ends in the sub-stent close to each other to form a wrapped portion, wherein several wrapped portions are formed on the sub-stent; and

(3) disposing a wrapping connector at a periphery of the wrapped portion, enabling two end portions of the wrapping connector to be firmly connected to regions corresponding to two ends of the wrapped portion, wrapping two wire tail ends of the wrapped portions by using the wrapping connector, and enabling the wrapped portion to be disposed at a distance from an edge of at least one end portion of the sub-stent.

Further, in step (3), the two wire tail ends penetrate into a wire mesh formed by a first wire extending along a first spiral direction and at least a second wire extending along a second spiral direction to form the wrapped portion.

Further, in step (2), the two end portions of the wrapping connector are fixed at the regions corresponding to the two ends of the wrapped portion through a coating process or a heat shrinking process to form firm connections.

Finally, it should be noted that the foregoing embodiments are merely intended to describe the technical solutions of this application, and shall not be construed as limitation. Although this application is described in detail with reference to the foregoing embodiments, one of ordinary skills in the art may understand that modifications still may be made to the technical solutions disclosed in the foregoing embodiments, or equivalent replacements may be made to some or all of the technical features. However, these modifications or equivalent replacements do not deviate from the nature of corresponding technique solutions from the scope of the technique solutions of the embodiments of this application.

What is claimed is:

1. An intraluminal stent, wherein the intraluminal stent comprises at least one sub-stent; the sub-stent has a radial compression form and a radial expansion form; the sub-stent has, in an axial direction, end portion regions at two ends and an intermediate region extending between the end portion regions; the sub-stent comprises at least a first wire extending along a first spiral direction and at least a second wire extending along a second spiral direction; and the first wire and the second wire extend in different directions to form several wire intersection points;
wherein the intraluminal stent has several wrapped portions for wrapping two corresponding wire tail ends; a wrapping connector is disposed at a periphery of the wrapped portion; two end portions of the wrapping connector are firmly connected to regions corresponding to two ends of the wrapped portion; and the wrapped portion is disposed at a distance from an edge of at least one end portion of the sub-stent.

2. The intraluminal stent according to claim 1, wherein a separation distance between the wrapped portion and the edge of at least one end portion of the sub-stent is a, and a length of the sub-stent along the axial direction is L, wherein a/L is greater than 1/20.

3. The intraluminal stent according to claim 1, wherein the two wire tail ends of the wrapped portions are wrapped by the wrapping connector in a manner of being close to or in contact with each other.

4. The intraluminal stent according to claim 3, wherein the two wire tail ends of the wrapped portions are wrapped by using the wrapping connector in a manner that the two tail ends can move relative to each other.

5. The intraluminal stent according to claim 1, wherein radial compression resistance force of a corresponding portion of the wrapping connector is not smaller than 10 N.

6. The intraluminal stent according to claim 1, wherein the wrapping connector is an elastic coating layer and/or a heat-shrinkable tube.

7. The intraluminal stent according to claim 1, wherein a length of the wrapping connector at least completely covers the wrapped portion, and the two corresponding wire tail ends in the wrapped portion are in contact with each other in an abutting manner or have an overlapping region.

8. The intraluminal stent according to claim 1, wherein at least one end portion region of the stent contracts radially when closing to the intermediate region, the wrapped portion is located at a radial contraction portion, and the wrapping connector is located at a radial contraction region.

9. The intraluminal stent according to claim 1, wherein a cross section of the sub-stent is circular, D-shaped, or elliptical.

10. The intraluminal stent according to claim 1, wherein the intraluminal stent is an L-shaped or a Y-shaped stent.

11. The intraluminal stent according to claim 1, wherein the tail end of the first wire extending along the first spiral direction is bent towards the intermediate region at the end portion region on at least one end of the sub-stent to form a bent portion, and is close to the corresponding tail end of the second wire extending along the second spiral direction to form a wrapped portion.

12. The intraluminal stent according to claim 1, wherein between two sub-stents, a wire tail end of one sub-stent extends towards a corresponding wire tail end of another sub-stent to form a wrapped portion.

13. The intraluminal stent according to claim 1, wherein a length of the wrapping connector is twice of a width of a braided mesh; in another spiral direction, there is a first wire or a second wire that crosses the wrapping connector; the braided mesh is a grid formed by means that the first wire intersects with the second wire; and said another spiral direction is a first spiral direction or a second spiral direction that is not provided with a wrapping connector.

14. The intraluminal stent according to claim 13, wherein both ends of the wrapping connector are located at the wire intersection points of the first wire and the second wire, and are clamped with the first wire or the second wire that is in said another spiral direction and forms the wire intersection points.

15. The intraluminal stent according to claim 13, wherein in the wrapping connector, a length for which the two wire tail ends overlap with each other is twice of the width of the braided mesh.

16. The intraluminal stent according to claim 1, wherein the wrapping connector is located in a transition region between the end portion region and the intermediate region of the sub-stent.

17. The intraluminal stent according to claim 1, wherein the wrapping connector is a heat-shrinkable tube, and a heat shrinkage ratio of the heat-shrinkable tube is greater than or equal to 30%.

18. A method for preparing the intraluminal stent according to claim 1, comprising the following steps:
(1) providing an intraluminal stent that comprises at least one sub-stent;
(2) making two corresponding wire tail ends in the sub-stent close to each other to form a wrapped portion, wherein several wrapped portions are formed on the sub-stent; and
(3) disposing a wrapping connector at a periphery of the wrapped portion, enabling two end portions of the wrapping connector to be firmly connected to regions corresponding to two ends of the wrapped portion, wrapping two wire tail ends of the wrapped portions by using the wrapping connector, and enabling the wrapped portion to be disposed at a distance from an edge of at least one end portion of the sub-stent.

19. The method for preparing the intraluminal stent according to claim 18, wherein in step (3), the two wire tail ends penetrate into a wire mesh formed by a first wire extending along a first spiral direction and at least a second wire extending along a second spiral direction to form the wrapped portion.

20. The method for preparing the intraluminal stent according to claim 18, wherein in step (2), the two end portions of the wrapping connector are fixed at the regions corresponding to the two ends of the wrapped portion through a coating process or a heat shrinking process to form firm connections.
